(19) United States
(12) Patent Application Publication
(10) Pub. No.: US 2002/0028438 A1
(43) Pub. Date: Mar. 7, 2002

(54) Diagnostic method for detecting an antigen or antibody in a blood sample

(75) Inventors: Hyeong Soon Shin, Seoul (KR); Kwang Soon Shin, Taejon-shi (KR); Tae Ryeon Heo, Seoul (KR); Hong Keun Chung, Seoul (KR)

Correspondence Address: HOWARD & HOWARD, The Pinehurst Office Center, Suite 101, 39400 Woodward Avenue, Bloomfield Hills, MI, 48304-5151, US

(73) Assignee: Vendia Tech Co. Ltd.

(21) Appl. No.: 09/924,288

(22) Filed: Aug. 8, 2001

Related U.S. Application Data
(62) Division of application No. 09/608,171, filed on Jun. 30, 2000.

(30) Foreign Application Priority Data
Aug. 23, 1999 (KR) 1999-35048
Feb. 3, 2000 (KR) 2000-5447

(51) Int. Cl. C12Q 1/70; G01N 33/567

(52) U.S. Cl. 435/5; 435/7.21

(57) ABSTRACT

The present invention relates to a method of bleaching blood samples, a diagnostic method and a diagnostic kit using whole blood samples. More particularly, this invention relates to the method of bleaching blood samples employing oxidant such as hydrogen peroxide, hypochlorite, permanganate, bichromate and nitrite, the diagnosis method for detecting antigen or antibody in blood samples and the diagnostic kit employing the above bleaching method and the conventional immunochromatographic assay.

### BACKGROUND OF THE INVENTION

1. Field of the Invention

The present invention relates to a method of bleaching blood samples, a diagnostic method and a diagnostic kit using whole blood samples. More particularly, this invention relates to a method of bleaching blood samples employing oxidant, etc., a diagnosis method for detecting infection with certain pathogen including hepatitis virus and a diagnostic kit embodying the above method.

2. Description of the Related Art

Immunochromatographic Assay (hereinafter referred to as “ICA”) is also referred to as “rapid test” due to its rapidity and simplicity. In such assay, tracer antibody molecules conjugated with gold particles bind to a particular antigen contained in a serum sample, after which the formed complexes pass through microspores of nitrocellulose membrane (hereinafter referred to as “NC membrane”) in terms of capillary phenomenon. The complexes finally bind to capture antibodies immobilized on the inner surface of microspore of the NC membrane and develop color of a positive line, whereby determining easily the existence of a particular antigen in the serum sample with the naked eye.

As noted above, the ICA, owing to simplicity of procedure and rapidity of the running result, has been widely used for the detection of various analytes such as hormones (Laitinen, M. P. et al., Acta. Chem. Scand. 50, 141-147(1996)), antigens (Sato, K. et al., J. Clin. Microbiol. 34, 1420-1423(1996)), antibodies (Vaughn, D. W. et al., J. Clin. Microbiol. 36, 234-238(1998)) and drugs (Habib, M. P. et al., Chest 92,129-134(1987)).

Furthermore, many diagnostic kits embodying the ICA have been currently produced on a commercial basis and enable alleged-patients themselves to determine or monitor a variety of conditions or disorders at home.

The present inventors already have developed the ICA kit for detecting hepatitis B surface antigen (hereinafter referred to as “HBsAg”). The details of the ICA kit are disclosed in Hyeong-Soon Shin et al., J. Korean Soc. Virology, 27, No. 2, 137-141(1997)

There are two major constituents in the ICA kit. One is the NC membrane which has two invisible lines on the surface and the other is a glass fiber filter containing antibody-gold particle conjugates in a dry state on the surface. Two kinds of antibodies, that is, the monoclonal anti-HBs being specific to antigen to be detected and Goat anti-mouse IgG, are immobilized on the lower line and the upper line of the NC membrane, respectively.

A sample is added to a sample well of the ICA kit and then the antibody-gold particle conjugates on the surface of the NC membrane in a dry state are rehydrated and then bound to antigens in the serum sample, after which the formed complexes pass through microspores of the NC membrane in terms of capillary phenomenon.

Thereinafter, the antigens of the complexes are reacted with the monoclonal anti-HBs immobilized on the lower line, resulting in developing a color. In addition, the upper line develops a color because the Goat anti-mouse IgG immobilized on the upper line may react with the antibody-gold particle conjugates although no antigen is present, thus the upper line always develops a color in each run of the test and may serve as a control line. That is to say, when antigens exist in the serum sample, both the positive line and the control line of the ICA kit become visible but only the control line becomes visible, when no antigen is present.

Meanwhile, whole blood can not be used in the ICA kits due to the visual hindrance of the color of red blood cells (RBCs). Hence, the ICA kit currently employs clear serum as a sample to be tested, which has to be previously subjected to coagulation and centrifugation to separate the blood cells. The above pretreatment process reduces less rapidity and simplicity of the ICA kit because additional time and machines are required for coagulation and separation to prepare serum after collecting whole blood.

In order to solve the aforementioned problems, a blood separation filter for blocking blood cells, which prohibits blood cells in the whole blood from moving across and ensures only filtered serum to be developed, has been adapted to the kit (Pall Corporation, WO 960314; and Boehringer Mannheim, EP 586789). However, the filter retards the development of serum and some of samples sometimes do not run because of blocking the sample well by clotting of intact whole blood in the well. Furthermore, the drier the applied sample serum becomes after developing through the NC membrane, the higher the concentration of salts of whole blood in the sample well becomes and finally inducing rupture of the red blood cells. The intracellular materials including the red pigment may move across the filter and cover the NC membrane to prevent the correct reading.

Consequently, there remains an urgent need in the art for a method to treat whole blood samples to apply to the ICA kit.

### SUMMARY OF THE INVENTION

To overcome the above mentioned shortcomings, the inventor et al. have made intensive studies and as a result developed a method of bleaching blood samples and adopted it to the conventional ICA kit, noting that the ICA kit may detect directly a variety of antigens or antibodies in whole blood samples without reducing its simplicity and rapidity. Therefore, this invention has been developed.

Accordingly, an object of this invention is to provide a method of bleaching blood samples.

Another object of this invention is to provide a method for detecting antigen or antibody in blood samples employing an existing ICA and the present method of bleaching blood samples.

Still another object of this invention is to provide an ICA kit being able to use non-separated blood as sample.

Other objects and advantages of this invention will become more apparent from the detailed description to follow taken in conjunction with the appended claims.

### BRIEF DESCRIPTION OF THE DRAWINGS

FIG. 1 is a graph showing the scanning profiles of the blood suspension bleached by this invention and one untreated.

FIG. 2 is a photograph representing the effect of various concentrations of hydrogen peroxide on whole blood.

### DETAILED DESCRIPTION OF THE INVENTION

This invention relates to a method of bleaching blood samples comprising a step of adding blood sample to pretreating solution containing oxidant including hydrogen peroxide, hypochlorite, permanganate, bichromate, nitrite, etc.

This invention also relates to a method for detecting antigen or antibody in blood samples employing the above method of bleaching blood samples and an existing ICA.

This invention also relates to an ICA kit comprising (a) a sample well having a glass fiber filter containing conjugates of gold particle and antibody specific to antigen to be detected; and (b) a strip having a nitrocellulose membrane containing a positive line with antibody to antigen to be detected and a control line with anti-mouse antibody, wherein the improvement further comprises a blood pretreating well containing a pretreating solution including oxidant such as hydrogen peroxide, hypochlorite, permanganate, bichromate and nitrite.

This invention is explained in more detail as set forth hereunder:

The oxidant used in this invention preferably includes, but not limited to, hydrogen peroxide, hypochlorite, permanganate, bichromate and nitrite. More preferably, the oxidant includes hydrogen peroxide, hypochlorite and permanganate and most preferably, hydrogen peroxide.

The reason why hydrogen peroxide is selected as the most preferable oxidant is that hydrogen peroxide may easily permeate across the membrane of red blood cell in which catalase catalyzes decomposition of hydrogen peroxide. Further to this, hydrogen peroxide may reduce the possibility of inadvertent infection due to sterilization action thereof, and some of the steps for preparing a sample may be eliminated, consequently ensuring safe treatment of blood samples.

As soon as a blood sample is added to pretreating solution, the hydrogen peroxide is decomposed to produce hydroxy radical, which degrade the porphyrin ring of hemoglobin molecule in red blood cell to release iron ion from hemoglobin, consequently bleaching the red blood cell.

The amount of hydrogen peroxide is preferably in the range of 30 to 0.01%; if the amount is less than 0.01%, the extent of bleaching is negligible due to the drop of oxidizing power. The amount of hydrogen peroxide may not exceed 30%, because commercial hydrogen peroxide commonly has a concentration of 30%.

According to the preferred embodiment of this invention, it is preferred that an antifoam is further added in order to restrain the mixture from vigorous foaming.

The antifoam employed in this invention advantageously includes, but not limited to, silicone-based antifoam such as a mixture of silicone oil and hydrophobic solid particles, hydrophobic silica, silicone oil and silicone polyether; alkyl alcohols with mono-hydroxyl group such as isopropyl alcohol; polyols such as polypropylene glycol; and surfactant such as Triton X-100 and Tween 80. More preferably, the antifoam is a mixture of silicone oil and graphite or a mixture of silicone oil and hydroxypropyl methyl cellulose substituted with methoxy groups.

It is preferred that the amount of antifoam is in the range of 0.01 to 1%; if the amount is less than 0.01%, the antifoaming activity is negligible; in the case of exceeding 1%, the hemolysis is generated due to increased interfacial activity.

Since hydrogen peroxide permeated into RBCs is decomposed to water and oxygen molecule, it is preferred to add further enzyme inhibitor in order to restrain from producing oxygen being responsible for vigorous foaming observed in the initial stage of the present invention.

The enzyme inhibitor used in this invention advantageously includes, but not limited to, cyanide, aminotriazole and sodium azide.

It is preferred that the amount of enzyme inhibitor is in the range of 0.0001 to 0.1%. If the amount is less than 0.0001%, the enzyme inhibition is not effective, resulting in undesirable foaming; if the amount exceeds 0.1%, most of cellular enzymes including catalase are completely inhibited, and thus a production of hydroxy radical is remarkably retarded, finally leading to a cripple reaction of bleaching.

As described above, both antifoam and enzyme inhibitor may be responsible for inhibition of vigorous foaming which results in restraining movement of a blood sample due to blocking of microspores of NC membrane.

According to the preferred embodiment of this invention, it is preferred to add further chelating agent so as to facilitate the reaction of bleaching. The chelating agent forms rapidly the complex compound with iron ion released from hemoglobin, thereby expediting the bleaching reaction of this invention. In addition to this, the chelating agent serves as an anticoagulant which prevents the collected blood sample from coagulating.

The chelating agent used in this invention includes, but not limited to, ethylenediaminetetraacetic acid, nitriloacetic acid, ethyleneglycol-bis(β-aminoethylether)-tetraacetic acid and 1,2-diaminocyclohexanetetraacetic acid.

The method for detecting antigen or antibody in a blood samples according to this invention employs conventional ICA and further comprises the step of bleaching the blood performed in such a manner that the blood sample is firstly added to a pretreating solution including oxidant such as hydrogen peroxide, hypochlorite, permanganate, bichromate and nitrite.

According to the method for detecting antigen or antibody of this invention, it is the most preferred to add hydrogen peroxide as oxidant.

In the preferred embodiment of this invention employing hydrogen peroxide as oxidant, an antifoam including silicone-based antifoam such as a mixture of silicone oil and hydrophobic solid particles, hydrophobic silica, silicone oil and silicone polyether; alkyl alcohols with mono-hydroxyl group such as isopropylalcohol; polyols such as polypropylene glycol; and surfactants such as Tween 80 and Triton X-100; and/or an enzyme inhibitor including cyanide, aminotriazole and sodium azide are further added to the blood sample.

According to this invention, it is preferred that a chelating agent including ethylenediaminetetraacetic acid, nitriloacetic acid, ethyleneglycol-bis(β-aminoethylether)-tetraacetic acid and 1,2-diaminocyclohexanetetraacetic acid is further added to the blood sample.

The method for detecting antigen or antibody in a blood samples according to this invention is applicable to a detection of a variety of antigens originated from infectious pathogens such as hepatitis B virus or incurable diseases such as colorectal cancer and hepatocarcinoma. The method is also applied to a detection of a variety of antibodies to antigens of hepatitis A virus, hepatitis C virus or human immunodeficiency virus (hereinafter, referred to as “HIV”), which are hard to scrutinize due to their low concentration in blood. The use of the method according to this invention has broader applicability and is not limited to the above examples.

According to the ICA kit of this invention, it is the most preferred that the oxidant contained in the pretreating solution is hydrogen peroxide.

In the case of employing hydrogen peroxide as oxidant, it is preferred that an antifoam including silicone-based antifoam such as a mixture of silicone oil and hydrophobic solid particles, hydrophobic silica, silicone oil and silicone polyether; alkyl alcohols with mono-hydroxyl group such as isopropyl alcohol; polyols such as polypropylene glycol; and surfactants such as Tween 80 and Triton X-100; and/or an enzyme inhibitor including cyanide, aminotriazole and sodium azide are further added to the pretreating solution in the pretreating well.

More preferably, the antifoam is a mixture of silicone oil and hydrophobic solid particles; and a mixture of silicone oil and graphite or a mixture of silicone oil and methyl cellulose substituted with methoxy groups is the most preferred one.

According to this invention, it is preferred that a chelating agent including ethylenediaminetetraacetic acid, nitriloacetic acid, ethyleneglycol-bis(β-aminoethylether)-tetraacetic acid and 1,2-diaminocyclohexanetetraacetic acid is further added to the pretreating solution in the pretreating well.

The pretreating well may be separately equipped with respect to other elements of the kit or joined to the cassette housing having nitrocellulose membrane.

According to the preferred embodiment of this invention, the kit further comprises pipetting means for treating or transferring a blood sample under consideration of a user's convenience.

Meanwhile, the detection of antibody in blood may be carried out with the kit of this invention in the case that this invention is subject to modifications being apparent to those skilled in the art. That is to say, the kit for detecting antibody may be reconstituted in such a manner that particular antigen specific to antibody to be detected are conjugated with the gold particles and immobilized on the test line.

The diagnostic kit according to this invention may be used for diagnosing a variety of conditions or disorders in mammals including human beings caused by hepatitis A virus, hepatitis B virus, hepatitis C virus or HIV but is not limited to the above.

As understood from the above, the diagnostic kit of this invention ensures the ICA kit to use whole blood as an applied sample without the coagulating and the centrifuging procedures of a blood sample, which are necessary steps in a conventional ICA kit. Consequently, the kit of this invention secures a personal diagnosis of a variety of diseases as mentioned above.

The following specific examples are intended to be illustrative of the invention and should not be construed as limiting the scope of the invention as defined by appended claims.

### EXAMPLE 1

Selection of Preferred Oxidant

In order to test the bleaching action of the oxidants used in this invention, the following tests were carried out:

Whole blood of 10 μl were added to 190 μl of PBS including 3% H₂O₂, 0.01M NaOCl or 0.01M KMnO₄, respectively, and then the resulting mixtures were incubated for 1 min. at room temperature, after which the bleaching patterns of the blood sample were observed.

The color of the blood sample treated with H₂O₂ was rapidly changed to white without agitation but the color of samples treated with NaOCl or KMnO₄ were slowly changed to dark brown after agitation.

Therefore, H₂O₂ was determined as the most suitable oxidant of this invention. It is speculated that the catalase existing in blood cells, which may decompose H₂O₂, is responsible for such a result and no enzyme to decompose NaOCl or KMnO₄ is present.

Determination of Suitable Concentration of Oxidant

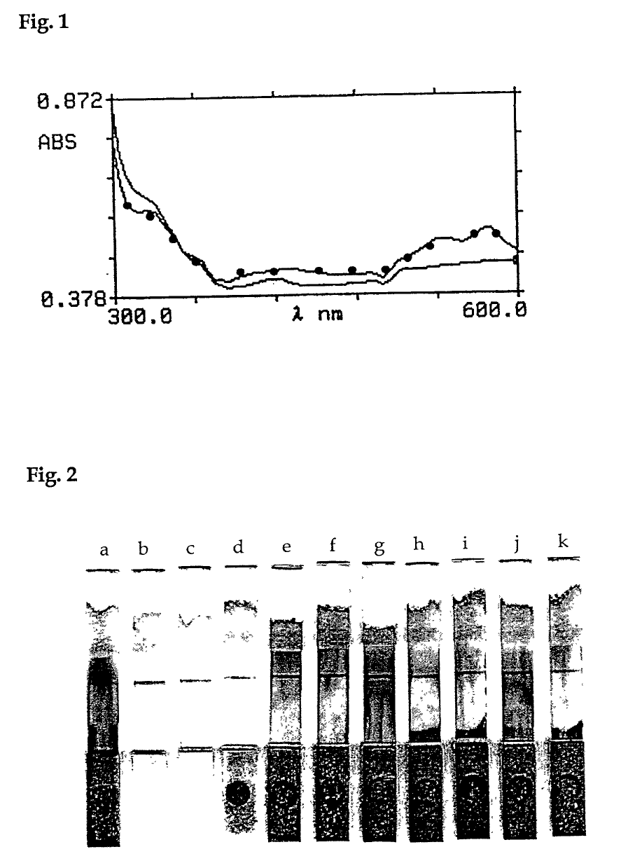

To determine the proper concentration of H₂O₂, PBS including 3% H₂O₂, were diluted with the same buffer by the 2-fold serial dilution to prepare 1.5, 0.75, 0.375, 0.188, 0.094, 0.047, 0.023, 0.01 and 0.005% H₂O₂solutions. Then, to 190 μl of each dilution, 10 μl of the whole blood were added, incubated for 1 min. at room temperature and then the bleaching patterns of the whole blood sample were observed, which are shown in FIG. 1.

In FIG. 1, (a) lane is a control representing the sample without treatment of H₂O₂; (b) lane represents the sample treated with 3% H₂O₂; (c) lane 1.500% H₂O₂; (d) lane 0.75% H₂O₂; (e) lane 0.375% H₂O₂; (f) lane 0.188% H₂O₂; (g) lane 0.094% H₂O₂; (h) lane 0.047% H₂O₂; (i) lane 0.023% H₂O₂; (j) lane 0.01% H₂O₂and (k) lane 0.005% H₂O₂.

As shown in FIG. 1, the samples treated with less than 0.01% H₂O₂ show a negligible degree of bleaching due to weaker oxidizing power but in the case of more than 0.01% H₂O₂, significant patterns of bleaching were observed. Therefore, the preferred concentration of H₂O₂was determined to be in the range of 30 to 001%.

Selection of Preferred Antifoam & Determination of Suitable Concentration Thereof

To select the preferred antifoam ensuring to restrain the mixture of the whole blood and the bleaching solution from vigorous foaming, various antifoams were examined as follows:

To 180 μl of PBS containing 1.5% H₂O₂, 10 μl of 5% Sag 471 (a mixture of silicone oil and hydrophobic solid particles; Osi specialities, USA), isopropyl alcohol or polypropylene glycol were added and 10 μl of the whole blood was added to each of the resulting mixtures to observe the antifoaming effect. The most suitable antifoam was determined to be Sag 471.

To determine the suitable concentration of antifoam, PBS containing 1.5% H₂O₂ and Sag 471 at the concentration of 0.25, 0.125, 0.0625, 0.0313, 0.0156, 0.0078, 0.0039, 0.002 and 0.001% were prepared, respectively. To 190 μl of each of the prepared solutions, 10 μl of the whole blood were added and incubated to observe bleaching and antifoaming effects. The preferred concentration of the antifoam was determined to be in the range of 0.001 to 1%.

Determination of Suitable Concentration of Enzyme Inhibitor

To determine the suitable concentration of enzyme inhibitor ensuring to restrain the mixture of the whole blood and the bleaching solution from vigorous foaming, actions of NaN₃ at various concentration were examined as follows:

PBS containing 0.05% NaN₃ (Sigma, USA) and 1.5% H₂O₂ was diluted with PBS containing 1.5% H₂O₂ by the 2-fold serial dilution method to prepare solutions containing 0.025%, 0.0125%, 0.006%, 0.003%, 0.0016%, 0.0008%, 0.0004% and 0.0002% NaN₃, respectively. Thereafter, 10 μl of the whole blood were added to 190 μl of each dilution to observe the effect on bleaching and antifoaming reactions. The preferred concentration of enzyme inhibitor was determined to be in the range of 0.0001 to 0.1%.

Evaluation on Effect of Anticoagulant

The whole blood sample employed in this invention is commonly treated with anticoagulant so as to inhibit the blood to coagulate during collecting a blood sample. The common anticoagulant including heparin, citrate and ethylenediaminetetraacetic acid (hereinafter, referred to as “EDTA”) were examined to evaluate the effect on bleaching reaction as follows:

To 1 ml of the whole blood sample, 0.1% heparin (Sigma, USA), 1M citrate or 1M EDTA were immediately added after collecting. Then, 10 μl of the anticoagulant-treated blood samples containing each anticoagulant were added to 190 μl of PBS containing 0.003% NaN₃, 0.016% Sag471 and 1.5% H₂O₂ in order to observe the effect on bleaching reaction.

The EDTA expedited the bleaching reaction by chelating iron ion released from hemoglobin. Namely, the chelating reaction by the EDTA serves as a driving force of the bleaching reaction. Therefore, the EDTA was determined to be the most preferred anticoagulant.

Evaluation on Effect of Temperature on Bleaching Activity

In order to examine the effect of the reaction temperature on the bleaching reaction, 190 μl of pretreating solution (PBS containing 1.5% H₂O₂, 0.016% Sag471, 0.003% NaN₃ and 5 mM EDTA) were added to 10 μl of the whole blood sample and reacted at 8° C., room temperature and 37° C., respectively.

The bleaching reaction was very slightly effected at 8° C., but significantly effected at room temperature and 37° C.

Determination of Preferred Amount of Sample Applied

In order to determine a suitable amount of blood sample, various amounts of a blood sample (10, 20, 30 and 40 μl,) were added to 190, 180, 170, and 160 μl of the pretreating solution (PBS containing 1.5% H₂O₂, 0.016% Sag471, 0.003% NaN₃ and 5 mM EDTA) respectively, after which the patterns of bleaching were observed.

The bleaching reaction was completely effected at less than 30 μl of blood sample and slightly effected at 40 μl of blood sample, however, the reaction was enough to read the result even when RBCs were not fully decolorized on a sample pad on the case of 40 μl of whole blood. In conclusion, the lower the volume of blood sample added to the pretreating solution, the more intense the degree of decolorization was on the case of applying less than 20 vol % to the pretreating solution. Therefore, the preferred amount of sample is approximately 20 vol % based on the amount of the pretreating solution. It is noted that the amount of blood sample to be applied may be increased in proportion to the amount of other components of the pretreating solution.

### EXAMPLE 2

Blood samples of 10 μl were added to the preferred pretreating solution (PBS containing 1.5% H₂O₂, 0.016% Sag471, 0.003% NaN₃ and 5 mM EDTA) and then was incubated for 1 min. at room temperature, after which the degree of bleaching was measured with a spectrophotometer (TIDAS, J&M Co., Germany).

As shown in FIG. 2, the absorbancy of the treated blood sample (------) was markedly reduced between 520 and 600 nm but somewhat increased between 300 and 320 nm, compared with that of the untreated (●---●).

### EXAMPLE 3

The ICA kit of this invention to detect HBsAg was manufactured as follows:

The kit was designed to have two major parts in the form of a cassette. In addition to this, the kit comprises an eppendorf tube as said blood pretreating well containing pretreating solution and spuit for transferring a blood sample.

NC membrane with microspores having a size of 0.8 μm was installed in a strip part among two major parts and then a positive line with immobilized polyclonal antibody to HBsAg (hereinafter, referred to as “anti-HBs”; Sigma, USA) was constructed on the lower part of NC membrane, after which a control line with immobilized Goat anti-mouse IgG (Sigma, USA) was constructed on the upper part of NC membrane.

Anti-HBs.gold particle conjugates deposited on glass fiber filter in dry state were prepared as follows: 6 μg of monoclonal anti-HBs (Sigma, USA) were added to 1 ml of the gold particle suspension (BBI, UK) and mixed gently for 10 min. Then, the resulting anti-HBs.gold particle conjugate was blocked with bovine serum albumin (hereinafter, referred to as “BSA”) for 10 min., collected by centrifugation (at 11,000 g for 1 hr. 4° C.) and washed twice with 50 mM Tris-HCl buffer (pH 8.0) containing 5% BSA and 0.1% polyethylene glycol. Finally, the conjugate was resuspended in the same buffer and Abs₅₈₀ of the working suspension was 5.

A glass fiber filter (Milipore, USA) installed in contact with the bottom part NC membrane was soaked in the anti-HBs.gold particle conjugate suspension and dried for 3 hrs. at 37° C.

160 μl of the pretreating solution (PBS containing 1.5% H₂O₂, 0.016% Sag471, 0.003% NaN₃and 5 mM EDTA) were added into the eppendorf tube and the opening of the tube was closed with a plug for preventing the solution from leaking during storage and conveyance of the kit.

### EXAMPLE 4

The detection of HBsAg in human blood was performed using the kit of this invention manufactured as EXAMPLE 3 as follows:

The blood samples were collected from alleged-patients having been hospitalized in the Hospital of the Koryo Medical College and then applied to ELISA (Enzygnost HBsAg, Boehringer Mannheim, Germany) to diagnose hepatitis B.

Then, 30-40 μl of the tested blood were added to the eppendorf tube containing the pretreating solution, incubated to expedite a bleaching reaction for 1 min. and the bleached blood sample was applied into the sample well using spuit. Thereafter, the rate of running and the patterns of color development of the positive line and the control line were observed.

After 2 min., the positive line became visible and the color became darker with the lapse of time. After 3 min., the control line became visible, reddish particles still moved, indicating that the reaction was not completed yet and this reaction proceeded to 5 min. It was recognized that the positive reaction started after 2 min. and was completed after about 5 min.

As mentioned above, this invention relates to a method of bleaching blood samples. Also, this invention relates to a method for detecting antigen or antibody in a blood samples employing an existing ICA and the present method of bleaching blood samples. This invention relates further to an ICA kit being able to use intact whole blood as a sample.

The diagnostic method and the kit of this invention may eliminate the steps of coagulation and centrifugation which are necessary in the conventional ICA kits using serum, and thus in the case of using whole blood samples, its rapidity and simplicity are not reduced.

In addition, this invention may be used by alleged-patients themselves for at-home monitoring of many conditions or disorders caused by hepatitis A virus, hepatitis B virus, hepatitis C virus, HIV, etc. The diagnostic kit of this invention may be steadily maintained in room-temperature storage and manufactured in a portable form, thereby being applicable wherever a diagnosis is required.

What is claimed is:

1. A method of bleaching blood samples comprising a step of adding blood sample to pretreating solution containing oxidant selected from the group consisting of hydrogen peroxide, hypochlorite, permanganate, bichromate and nitrite.

2. The method according to claim 1, wherein the oxidant is hydrogen peroxide.

3. The method according to claim 2, wherein the amount of hydrogen peroxide is in the range of 30 to 0.01%.

4. The method according to claim 3, wherein the method further comprises the step of adding antifoam selected from the group consisting of silicone based-antifoam selected from the group consisting of a mixture of silicone oil and hydrophobic solid particles, hydrophobic silica, silicone oil and silicone polyether; alkyl alcohols; polyols; and surfactants.

5. The method according to claim 4, wherein the mixture of silicone oil and hydrophobic solid particles is a mixture of silicone oil and graphite or a mixture of silicone oil and hydroxypropyl methyl cellulose substituted with methoxy groups.

6. The method according to claim 4, wherein the method further comprises the step of adding enzyme inhibitor selected from the group consisting of cyanide, aminotriazole and sodium azide.

7. The method according to claim 1 , wherein the method further comprises the step of adding chelating agent selected from the group consisting of ethylenediaminetetraacetic acid, nitriloacetic acid, ethyleneglycol-bis(β-aminoethylether)-tetraacetic acid and 1,2-diaminocyclohexanetetraacetic acid.

8. A method for detecting antigen or antibody in blood samples employing immunochromatographic assay, characterized in that the method further comprises the step of bleaching the blood performed in such a manner that the blood sample is firstly added to a pretreating solution containing oxidant selected from the group consisting of hydrogen peroxide, hypochlorite, permanganate, bichromate and nitrite.

9. The method according to claim 8, wherein the pretreating solution comprises 30 to 0.1% of hydrogen peroxide as oxidant.

10. The method according to claim 9, wherein the pretreating solution further comprises antifoam selected from the group consisting of silicone based-antifoam selected from the group consisting of a mixture of silicone oil and hydrophobic solid particles, hydrophobic silica, silicone oil and silicone polyether, alkyl alcohols, polyols and surfactants; enzyme inhibitor selected from the group consisting of cyanide, aminotriazole and sodium azide; and chelating agent selected from the group consisting of ethylenediaminetetraacetic acid, nitriloacetic acid, ethyleneglycol-bis(β-aminoethylether)-tetraacetic acid and 1,2-diaminocyclohexanetetraacetic acid.

11. The method according to any one of claims 8 to 10, wherein the method is used for detecting antigen or antibody to hepatitis A virus, hepatitis B virus, hepatitis C virus or human immunodeficiency virus.

12. In an immunochromatographic assay kit comprising (a) a sample well having a glass fiber containing conjugate of gold particle and antibody specific to antigen to be detected; and (b) a strip having nitrocellulose membrane containing a positive line with antibody to antigen to be detected and a control line with anti-mouse antibody, the improvement which further comprises a blood pretreating well containing a pretreating solution including oxidant selected from the group consisting of hydrogen peroxide, hypochlorite, permanganate, bichromate and nitrite.

13. The immunochromatographic assay kit according to claim 12, wherein the oxidant contained in the pretreating solution is hydrogen peroxide.

14. The immunochromatographic assay kit according to claim 13, wherein the pretreating solution further comprises antifoam selected from the group consisting of silicone-based antifoam selected from the group consisting of a mixture of silicone oil and hydrophobic solid particles, hydrophobic silica, silicone oil and silicone polyether; alkyl alcohols; polyols and surfactants.

15. The immunochromatographic assay kit according to claim 14, wherein the mixture of silicone oil and hydrophobic solid particles is a mixture of silicone oil and graphite or a mixture of silicone oil and methyl cellulose substituted with methoxy groups.

16. The immunochromatographic assay kit according to claim 14, wherein the pretreating solution further comprises enzyme inhibitor selected from the group consisting of cyanide, aminotriazole and sodium azide.

17. The immunochromatographic assay kit according to claim 16, wherein the pretreating solution further comprises chelating agent selected from the group consisting of ethylenediaminetetraacetic acid, nitriloacetic acid, ethyleneglycol-bis(β-aminoethylether)-tetraacetic acid and 1,2-diaminocyclohexanetetra acetic acid.

18. The immunochromatographic assay kit according to claim 12, wherein the blood pretreating well is separately equipped or joined to a cassette housing having the assay strip.

19. The immunochromatographic assay kit according to claim 12, wherein the kit further comprises a pipetting means for transferring a blood sample.

20. The immunochromatographic assay kit according to claim 12,
wherein the kit is used for diagnosing a variety of conditions or disorders caused by hepatitis A virus, hepatitis B virus, hepatitis C virus or human immunodeficiency virus.
